(19) United States
(12) Patent Application Publication
(10) Pub. No.: US 2025/0064715 A1
(43) Pub. Date: Feb. 27, 2025

(54) Oral Rinse Formulations Having Essential Oils

(71) Applicant: ProSANUS LLC, New York, NY (US)

(72) Inventor: Kimberly S. Kalaja, New York, NY (US)

(21) Appl. No.: 18/682,255

(22) PCT Filed: Aug. 4, 2022
(86) PCT No.: PCT/US2022/039403

§ 371 Date: Feb. 8, 2024

Related U.S. Application Data
(60) Provisional application No. 63/260,129, filed on Aug. 10, 2021.

(51) Int. Cl. A61K 8/92; A61Q 11/00

(52) U.S. Cl. CPC A61K 8/922; A61Q 11/00

(57) ABSTRACT

An oral-care rinse composition comprises water, thyme essential oil, lemongrass essential oil, and peppermint essential oil. The water is present in an amount that is greater than 95-99 weight % based on the total weight of the rinse composition. The thyme essential oil is present in an amount of 0.15 to 0.65 weight % based on the total weight of the rinse composition. The lemongrass essential oil is present in an amount of 0.15 to 0.65 weight % based on the total weight of the rinse composition. The peppermint essential oil is present in an amount of 0.15 to 0.65 weight % based on the total weight of the rinse composition.

### RELATED MATTERS

The present application claims priority to U.S. Provisional Patent Application Ser. No. 63/260,129, entitled “Oral Rinse Formulations Having Essential Oils” and filed on Aug. 10, 2021, which is herein incorporated by reference in its entirety.

### TECHNICAL FIELD

The present disclosure general relates to oral rinse formulations that contains certain essential oils that assist with preventing plaque, oral malodor, and staining preferably without the need for alcohol, dyes, or other additives.

### BACKGROUND

Poor oral health adversely affects a person's general health. For instance, there is believed to be a correlation between poor oral health and diabetes, heart and lung diseases, strokes, and low birth weight. Accordingly, there is a need for compositions and related methods for enhancing oral health care.

Dental plaque adheres to surfaces of human teeth. Within the plaque, a reaction occurs by which inorganic salts become more adhesive, and calcification starts from a layer which touches the tooth surface. The calcification advances as the plaque becomes older and thicker, and new plaques adhere to the surface of the calcified plaques and causes calcification. Organic components contained in dental plaque include bacterial cells and endotoxins. Dental plaque is responsible for many of the diseases common to the oral cavity including dental caries, periodontitis, and gingivitis. It also creates problems for patients who have dental implants.

Several known oral rinses use alcohol and/or other anti-bacterial components, including some of the hundreds of known essential oils. However, there is a need to avoid the use of alcohol-base rinses as the alcohol can have detrimental effects to the user, such as dry mouth and pH imbalances in the mouth which can promote or increase bacterial and fungal growth. Further, plaque and tartar are often treated with abrasive ingredients in toothpastes and oral rinses which can be harmful to tooth enamel over time. Though essentials oils are natural products that may have antibacterial properties, they do not all act in the same manner to help retard bacterial growth, especially in the mouth. After several years of experimentation with various essential oils (including Lavender (Lavandula angustifolia), Cinnamon (Cinnamomum verum), Clove (Syzygium aromaticum), Eucalyptus Globulus (Eucalyptus globulus), Eucalyptus Radiata (Eucalyptus Radiata), Ginger (Zingiber officinale), Lemon (Citrus limon), Lemongrass (Cymbopogon flexuosus), Lime (Citrus aurantifolia), Orange (Citrus sinensis), Tangerine (Citrus reticulata), Oregano (Origanum compactum), Rosemary (Rosmarinus officinalis CT cineol), Tea Tree (Melaleuca alternifolia), Thyme (Thymus vulgaris), Peppermint (Mentha piperita), Spearmint (Mentha spicata), Wintergreen (Gaultheris procumbens), the inventor has honed-in on specific formulations that she and her dental clinicians have observed as reducing dental plaque, oral malodor, and stains to the teeth.

There is an ongoing need exists for an alcohol-free, fluoride-free, chemical-free, abrasive-free, natural oral rinse that is non-irritating, safe, and can be repeatedly used. The oral-rinse formulations described below fill that need.

### SUMMARY

In one implementation, the present invention is an oral-care rinse composition, comprising water, thyme essential oil, lemongrass essential oil, and peppermint essential oil. The water is present in an amount that is greater than 95 weight % based on the total weight of the oral-care rinse composition. The thyme essential oil is present in an amount of 0.15 to 0.25 weight % based on the total weight of the oral-care rinse composition. The lemongrass essential oil is present in an amount of 0.15 to 0.25 weight % based on the total weight of the oral-care rinse composition. The peppermint essential oil is present in an amount of 0.15 to 0.25 weight % based on the total weight of the oral-care rinse composition. Preferably, the water is present in an amount that is greater than 97 or 99 weight % based on the total weight of the oral-care rinse composition.

In another implementation, the present invention is an oral-care rinse composition, comprising water, thyme essential oil, lemongrass essential oil, peppermint essential oil, and tea tree essential oil. The water is present in an amount that is greater than 95 weight % based on the total weight of the oral-care rinse composition. The thyme essential oil is present in an amount of 0.10 to 0.20 weight % based on the total weight of the oral-care rinse composition. The lemongrass essential oil is present in an amount of 0.10 to 0.20 weight % based on the total weight of the oral-care rinse composition. The peppermint essential oil is present in an amount of 0.15 to 0.25 weight % based on the total weight of the oral-care rinse composition. The tea tree essential oil is present in an amount of 0.05 to 0.10 weight % based on the total weight of the oral-care rinse composition. Preferably, the water is present in an amount that is greater than 97 or 99 weight % based on the total weight of the oral-care rinse composition.

In yet another implementation, the present invention is an oral-care rinse composition, comprising water, thyme essential oil, lemongrass essential oil, peppermint essential oil, and tea tree essential oil. The water is present in an amount that is greater than 99 weight % based on the total weight of the oral-care rinse composition. The thyme essential oil is present in an amount of about 0.15 weight % based on the total weight of the oral-care rinse composition. The lemongrass essential oil is present in an amount of about 0.15 weight % based on the total weight of the oral-care rinse composition. The peppermint essential oil is present in an amount of about 0.18 weight % based on the total weight of the oral-care rinse composition. The tea tree essential oil is present in an amount of about 0.08 weight % based on the total weight of the oral-care rinse composition.

In one implementation, the present invention is an oral-care rinse composition, comprising water, thyme essential oil, lemongrass essential oil, and peppermint essential oil. The water is present in an amount that is greater than 99 weight % based on the total weight of the oral-care rinse composition. The thyme essential oil is present in an amount of about 0.19 weight % based on the total weight of the oral-care rinse composition. The lemongrass essential oil is present in an amount of about 0.19 weight % based on the total weight of the oral-care rinse composition. The peppermint essential oil is present in an amount of about 0.19 weight % based on the total weight of the oral-care rinse composition.

In another implementation, the present invention is an oral-care rinse composition, comprising water, thyme essential oil, lemongrass essential oil, peppermint essential oil, and one or more additional essential oils. The water is present in an amount that is greater than 99 weight % based on the total weight of the oral-care rinse composition. The thyme essential oil is present in an amount of about 0.15 weight % based on the total weight of the oral-care rinse composition. The lemongrass essential oil is present in an amount of about 0.15 weight % based on the total weight of the oral-care rinse composition. The peppermint essential oil is present in an amount of about 0.18 weight % based on the total weight of the oral-care rinse composition. The one or more additional essential oils is present in an amount of less than about 0.2 weight % based on the total weight of the oral-care rinse composition. The one or more additional essential oils may include tea tree essential oil, clove essential oil, and/or spearmint essential oil.

In another implementation, the present invention is an oral-care rinse composition, comprising water, and combinations of thyme essential oil, lemongrass essential oil, peppermint essential oil, tea tree essential oil, clove essential oil, and/or spearmint essential oil. The water is present in an amount that is greater than 97 weight %, 98 weight %, or 99 weight % based on the total weight of the oral-care rinse composition.

In another implementation, the present invention is an oral-care rinse composition, comprising water and a combination of essential oils including thyme essential oil, lemongrass essential oil, and peppermint essential oil. The water is present in an amount that is greater than 99 weight % based on the total weight of the oral-care rinse composition. The combination of essential oils is in a collective amount that is between 0.3 weight % and 0.8 weight % based on the total weight of the oral-care rinse composition.

In a further implementation, the oral-care rinse composition comprises water in amount that is greater than 95 weight % based on the total weight of the oral-care rinse composition, thyme essential oil in an amount of from 0.15 to 0.65 weight % based on the total weight of the oral-care rinse composition, lemongrass essential oil in an amount of from 0.15 to 0.65 weight % based on the total weight of the oral-care rinse composition, and peppermint essential oil in an amount of from 0.15 to 0.65 weight % based on the total weight of the oral-care rinse composition.

In such an implementation, the thyme essential oil may be present in the amount of about 0.15 to 0.25 weight % based on the total weight of the oral-care rinse composition, the lemongrass essential oil may be present in the amount of about 0.15 to 0.25 weight % based on the total weight of the oral-care rinse composition, and the peppermint essential oil may be present in the amount of about 0.15 to 0.25 weight % based on the total weight of the oral-care rinse composition.

In a further implementation, the thyme essential oil may be present in the amount of about 0.25 to 0.65 weight % based on the total weight of the oral-care rinse composition, the lemongrass essential oil may be present in the amount of about 0.25 to 0.65 weight % based on the total weight of the oral-care rinse composition, and the peppermint essential oil may be present in the amount of about 0.25 to 0.65 weight % based on the total weight of the oral-care rinse composition. For example, the thyme essential oil is present in the amount of about 0.4 to 0.5 weight % based on the total weight of the oral-care rinse composition, the peppermint essential oil is present in the amount of about 0.4 to 0.5 weight % based on the total weight of the oral-care rinse composition, and the lemongrass essential oil is present in the amount of about 0.4 to 0.5 weight % based on the total weight of the oral-care rinse composition.

In a further aspect, the water is in amount that is greater than 98 or 99 weight % based on the total weight of the oral-care rinse composition.

In another aspect, an emulsifier and/or a stabilizing agent may be present.

In yet another aspect, the oral-care rinse composition includes one or more additional essential oils in a collective amount that is less than 0.5 weight % based on the total weight of the oral-care rinse composition, the one or more additional essential oils additional essential oil being tea tree essential oil, clove essential oil, and/or spearmint essential oil.

In a further aspect, the water is the only constituent other than the essential oils.

In a further implementation, an oral-care rinse composition, comprises water in amount that is greater than 98 weight % based on the total weight of the oral-care rinse composition, thyme essential oil in an amount of about 0.4 to 0.5 weight % based on the total weight of the oral-care rinse composition, lemongrass essential oil in an amount of about 0.4 to 0.5 weight % based on the total weight of the oral-care rinse composition, and peppermint essential oil in an amount of about 0.4 to 0.5 weight % based on the total weight of the oral-care rinse composition.

In yet a further implementation, an oral-care rinse composition comprises water in amount that is greater than 97 weight % based on the total weight of the oral-care rinse composition, and a combination of essential oils that includes one or more of thyme essential oil, lemongrass essential oil, peppermint essential oil, tea tree essential oil, clove essential oil, and/or spearmint essential oil.

In such an implementation, each of the thyme essential oil, the lemongrass essential oil, the peppermint essential oil may be present in the amount of about 0.25 to 0.65 weight % based on the total weight of the oral-care rinse composition.

In an alternative implementation, an oral-care rinse composition comprises water in amount that is greater than 97 weight % based on the total weight of the oral-care rinse composition and a combination of essential oils that includes at least thyme essential oil, lemongrass essential oil, and peppermint essential oil. The combination of essential oils being in a collective amount that is between 1.0 weight % and 3.0 weight % based on the total weight of the oral-care rinse composition.

In such an implementation, the thyme essential oil may be present in an amount that is less than 1.0 weight % based on the total weight of the oral-care rinse composition. The lemongrass essential oil may be present in an amount that is less than 1.0 weight % based on the total weight of the oral-care rinse composition. The peppermint essential oil is present in an amount that is less than 1.0 weight % based on the total weight of the oral-care rinse composition.

The present invention further contemplates methods of using the oral-care rinse compositions for reducing dental plaque, periodontitis (including gingivitis), oral malodor, and/or staining to the teeth. The present invention further contemplates methods of using the oral-care rinse compositions for whitening the teeth.

For example, the present invention includes a method of preventing stains to the teeth. The method comprises rinsing with an oral-care rinse composition at least once per day. The oral rinse composition includes (i) water in amount that is greater than 97, 98, or 99 weight % based on the total weight of the oral-care rinse composition, (ii) thyme essential oil in an amount of from 0.15 to 0.65 weight % based on the total weight of the oral-care rinse composition, (iii) lemongrass essential oil in an amount of from 0.15 to 0.65 weight % based on the total weight of the oral-care rinse composition, and (iv) peppermint essential oil in an amount of from 0.15 to 0.65 weight % based on the total weight of the oral-care rinse composition.

As another example, the present invention includes a method of reducing the growth of plaque on the teeth. The method comprises rinsing with an oral-care rinse composition at least once per day. The oral rinse composition includes (i) water in amount that is greater than 97, 98, or 99 weight % based on the total weight of the oral-care rinse composition, (ii) thyme essential oil in an amount of from 0.15 to 0.65 weight % based on the total weight of the oral-care rinse composition, (iii) lemongrass essential oil in an amount of from 0.15 to 0.65 weight % based on the total weight of the oral-care rinse composition, and (iv) peppermint essential oil in an amount of from 0.15 to 0.65 weight % based on the total weight of the oral-care rinse composition.

In a further example, the present invention includes a method of neutralizing malodor in the mouth. The method comprises rinsing with an oral-care rinse composition at least once per day. The oral rinse composition includes (i) water in amount that is greater than 97, 98, or 99 weight % based on the total weight of the oral-care rinse composition, (ii) thyme essential oil in an amount of from 0.15 to 0.65 weight % based on the total weight of the oral-care rinse composition, (iii) lemongrass essential oil in an amount of from 0.15 to 0.65 weight % based on the total weight of the oral-care rinse composition, and (iv) peppermint essential oil in an amount of from 0.15 to 0.65 weight % based on the total weight of the oral-care rinse composition.

In yet a further example, the present invention includes a method of whitening the teeth. The method comprises rinsing with an oral-care rinse composition at least once per day. The oral rinse composition includes (i) water in amount that is greater than 97, 98, or 99 weight % based on the total weight of the oral-care rinse composition, (ii) thyme essential oil in an amount of from 0.15 to 0.65 weight % based on the total weight of the oral-care rinse composition, (iii) lemongrass essential oil in an amount of from 0.15 to 0.65 weight % based on the total weight of the oral-care rinse composition, and (iv) peppermint essential oil in an amount of from 0.15 to 0.65 weight % based on the total weight of the oral-care rinse composition.

Within these methods, the water is the only constituent other than the essential oils.

Within these methods, the oral rinse composition may also include one or more additional essential oils in a collective amount that is less than 0.5 weight % based on the total weight of the oral-care rinse composition. The one or more additional essential oils additional essential oil being tea tree essential oil, clove essential oil, and/or spearmint essential oil

While the present disclosure is susceptible to various modifications and alternative forms, specific implementations have been shown by way of example in the drawings and will be described in further detail herein. It should be understood, however, that the present disclosure is not intended to be limited to the particular forms disclosed. Rather, the present disclosure is to cover all modifications, equivalents, and alternatives falling within the spirit and scope of the present disclosure as defined by the appended claims.

### DETAILED DESCRIPTION

The present invention provides formulations, and related methods, suitable for application onto the oral cavity and teeth wherein, after application of the formulations thereon, a reduction in the concentration of bacteria on the teeth and/or on the surrounding soft tissue. The reduced bacteria is believed to retard the development of dental plaque, of periodontitis (including gingivitis), of oral malodor, and of stains to the teeth. In some aspects, the formulations may also remove some of the existing staining already present on the teeth. The formulations in accordance with the present invention discussed below are preferably alcohol-free or have a minor amount of alcohol. The formulations in accordance with the present invention discussed below are preferably free from other additives or chemicals. The essential oils in the formulations are preferably organic essential oils.

### Oral-Rinse Formulation #1

Oral-rinse Formulation #1 has been used to reduce dental plaque, oral malodor, and of stains to the teeth. Oral-rinse Formulation #1 includes water, thyme essential oil, lemongrass essential oil and peppermint essential oil as the primary constituents. The essential oils have densities that are slightly lower than the density of water. Although each essential oil differs slightly in density with ranges typically from about 0.85 g/ml to about 0.95 g/ml, they average about 0.9 g/ml, which has been used for the calculations below for Oral-rinse Formulation #1.

TABLE 1
Oral-rinse Formulation #1.

| | | | | | Relative Weight | |
|---|---|---|---|---|---|---|
| Constituent | Amount | | Weight | | Percentage | |
| Water | 236.59 ml | | 236.59 | g | 99.43 | weight % |
| | (8 fl. oz.) | | | | | |
| Thyme Essential Oil | 0.5 | ml | 0.45 | g | 0.19 | weight % |
| Lemongrass Essential Oil | 0.5 | ml | 0.45 | g | 0.19 | weight % |
| Peppermint Essential Oil | 0.5 | ml | 0.45 | g | 0.19 | weight % |
| Total | 238.09 | ml | 237.94 | g | | |

Accordingly, in another embodiment based on Oral-rinse Formulation #1, the formulation includes an amount of thyme essential oil in an amount of from 0.15 to 0.25 weight % based on the total weight of the oral-care rinse composition, lemongrass essential oil in an amount of from 0.15 to 0.25 weight % based on the total weight of the oral-care rinse composition, and peppermint essential oil in an amount of from 0.15 to 0.25 weight % based on the total weight of the oral-care rinse composition. Thyme essential oil kills and retards the growth of pathogenic organisms that cause tooth decay, gingivitis and malodor. Peppermint essential oil also kills and retards the growth of bacteria and fungi. Lemongrass essential oil is believed to provide a synergistic effect for whitening and inhibiting plaque and tartar build-up. The formulation includes water as the primary constituent in an amount that is greater than 95 weight % based on the total weight of the oral-care rinse composition, greater than 97 weight % based on the total weight of the oral-care rinse composition, greater than 98 weight % based on the total weight of the oral-care rinse composition, or greater than 99 weight % based on the total weight of the oral-care rinse composition. The water may be the only constituent other than the essential oils. Although the water can be tap water, it is preferably deionized water or distilled water.

In another preferred embodiment, the oral rinse formulation includes about 99.4 weight % of water and about 0.2 weight % of each of thyme essential oil, lemongrass essential oil, and peppermint essential oil, such as formulation including about 0.19 weight % of each of thyme essential oil, lemongrass essential oil, and peppermint essential oil.

The formulations based on Formulation #1 may include other constituents, such as an emulsifier and/or a solubilizing agent (e.g., glycerin or lecithin) to help maintain the essential oils in suspension in the water. Glycerin is believed to assist in assisting solubilization and/or emulsification of organic ingredients in aqueous formulations, and may also provide a degree of sweetness as well as some antimicrobial properties to the formulation. When included in the formulation, the emulsifier and/or the solubilizing agent is preferably present in amount that is less than 0.15 weight % based on the total weight of the oral-care rinse composition, less than 0.10 weight % based on the total weight of the oral-care rinse composition, or less than 0.05 weight % based on the total weight of the oral-care rinse composition. However, the formulations do not require an emulsifier or a solubilizing agent. The formulations can be shaken in a bottle or container before use to distribute the oils evenly within the water.

The formulation may also include other essential oils as secondary constituents. When other essential oils (e.g., tea tree oil, clove oil, and/or spearmint oil) are present in the oral-rinse formulation, they are preferably present in amounts that are less than the amount of the thyme essential oil, lemongrass essential oil, and peppermint essential oil. For example, the additional essential oils may be present in amount that is less than 0.15 weight % based on the total weight of the oral-care rinse composition, less than 0.10 weight % based on the total weight of the oral-care rinse composition, or less than 0.05 weight % based on the total weight of the oral-care rinse composition.

In another formulation, the present invention is an oral-care rinse composition, comprising water and a combination of essential oils including thyme essential oil, lemongrass essential oil, and peppermint essential oil. The water is preferably in amount that is greater than 99 weight % based on the total weight of the oral-care rinse composition. The combination of essential oils being in a collective amount that is between 0.3 weight % and 0.8 weight % based on the total weight of the oral-care rinse composition. For example, the combination of essential oils can be in a collective amount that is about 0.3 weight %, about 0.4 weight %, about 0.5 weight %, about 0.6 weight %, about 0.7 weight %, and about 0.8 weight % based on the total weight of the oral-care rinse composition.

### Oral-Rinse Formulation #2

Oral-rinse Formulation #2 is similar to Formulation #1, but includes larger amounts of essential oils. Like Formulation, #1, Oral-rinse Formulation #2 has been used to reduce dental plaque, oral malodor, and of stains to the teeth. Oral-rinse Formulation #2 includes water, thyme essential oil, lemongrass essential oil and peppermint essential oil as the primary constituents. The essential oils have densities that are slightly lower than the density of water. Although each essential oil differs slightly in density with ranges typically from about 0.85 g/ml to about 0.95 g/ml, they average about 0.9 g/ml, which has been used for the calculations below for Oral-rinse Formulation #2.

TABLE 2
Oral-rinse Formulation #2.

| | | | | | Relative Weight | |
|---|---|---|---|---|---|---|
| Constituent | Amount | | Weight | | Percentage | |
| Water | 236.59 ml | | 236.59 | g | 98.62 | weight % |
| | (8 fl. oz.) | | | | | |
| Thyme Essential Oil | 1.2 | ml | 1.1 | g | 0.46 | weight % |
| Lemongrass Essential Oil | 1.2 | ml | 1.1 | g | 0.46 | weight % |
| Peppermint Essential Oil | 1.2 | ml | 1.1 | g | 0.46 | weight % |
| Total | 240.19 | ml | 239.89 | g | | |

Accordingly, in another embodiment based on Formulation #2, the formulation includes an amount of thyme essential oil in an amount of from 0.25 to 0.65 weight % based on the total weight of the oral-care rinse composition, lemongrass essential oil in an amount of from 0.25 to 0.65 weight % based on the total weight of the oral-care rinse composition, and peppermint essential oil in an amount of from 0.25 to 0.65 weight % based on the total weight of the oral-care rinse composition. Thyme essential oil kills and retards the growth of pathogenic organisms that cause tooth decay, gingivitis and malodor. Peppermint essential oil also kills and retards the growth of bacteria and fungi. Lemongrass essential oil is believed to provide a synergistic effect for whitening and inhibiting plaque and tartar build-up. The formulation includes water as the primary constituent in an amount that is greater than 95 weight % based on the total weight of the oral-care rinse composition, greater than 97 weight % based on the total weight of the oral-care rinse composition, greater than 98 weight % based on the total weight of the oral-care rinse composition, or greater than 99 weight % based on the total weight of the oral-care rinse composition. The water may be the only constituent other than the essential oils. Although the water can be tap water, it is preferably deionized water or distilled water.

In another preferred embodiment, the oral rinse formulation includes greater than 98 weight % of water and about 0.4 to 0.5 weight % of each of thyme essential oil, lemongrass essential oil, and peppermint essential oil, such as formulation including about 0.45 weight % of each of thyme essential oil, lemongrass essential oil, and peppermint essential oil.

The formulations based on Formulation #2 may include other constituents, such as an emulsifier and/or a solubilizing agent (e.g., glycerin or lecithin) to help maintain the essential oils in suspension in the water. Glycerin is believed to assist in assisting solubilization and/or emulsification of organic ingredients in aqueous formulations, and may also provide a degree of sweetness as well as some antimicrobial properties to the formulation. When included in the formulation, the emulsifier and/or the solubilizing agent is preferably present in amount that is less than 0.15 weight % based on the total weight of the oral-care rinse composition, less than 0.10 weight % based on the total weight of the oral-care rinse composition, or less than 0.05 weight % based on the total weight of the oral-care rinse composition. However, the formulations do not require an emulsifier or a solubilizing agent. The formulations can be shaken in a bottle or container before use to distribute the oils evenly within the water.

A formulation similar to Formulation #2 may also include other essential oils as secondary constituents. When other essential oils (e.g., tea tree oil, clove oil, and/or spearmint oil) are present in the oral-rinse formulation, they are preferably present in amounts that are less than the amount of the thyme essential oil, lemongrass essential oil, and peppermint essential oil. For example, the additional essential oils may be present in amount that is less than 0.15 weight % based on the total weight of the oral-care rinse composition, less than 0.10 weight % based on the total weight of the oral-care rinse composition, or less than 0.05 weight % based on the total weight of the oral-care rinse composition.

In another formulation, the present invention is an oral-care rinse composition, comprising water and a combination of essential oils including thyme essential oil, lemongrass essential oil, and peppermint essential oil. The water is preferably in amount that is greater than 97 weight % based on the total weight of the oral-care rinse composition. The combination of essential oils being in a collective amount that is between 0.8 weight % and 3.0 weight %, or preferably between 1.0 weight % and 2.0 weight % based on the total weight of the oral-care rinse composition. For example, the combination of essential oils can be in a collective amount that is about 0.8 weight %, about 0.9 weight %, about 1.0 weight %, about 1.1 weight %, about 1.2 weight %, about 1.3 weight %, about 1.4 weight %, about 1.5 weight %, about 1.6 weight %, about 1.7 weight %, about 1.8 weight %, about 1.9 weight %, and about 2.0 weight % based on the total weight of the oral-care rinse composition.

Considering both Oral-Rinse Formulations #1 and #2 collectively, the present invention contemplates formulations comprising water and a combination of essential oils including thyme essential oil, lemongrass essential oil, and peppermint essential oil. The formulation includes an amount of thyme essential oil in an amount of from 0.15 to 0.65 weight % based on the total weight of the oral-care rinse composition, lemongrass essential oil in an amount of from 0.15 to 0.65 weight % based on the total weight of the oral-care rinse composition, and peppermint essential oil in an amount of from 0.15 to 0.65 weight % based on the total weight of the oral-care rinse composition. The water in the formulation is the primary constituent and is present in an amount that is greater than 95 weight % based on the total weight of the oral-care rinse composition, greater than 97 weight % based on the total weight of the oral-care rinse composition, greater than 98 weight %, or greater than 99 weight %. The formulation may also optionally include one or more additional essential oils in a collective amount that is less than 0.5 weight % based on the total weight of the oral-care rinse composition. The one or more additional essential oils additional essential oil is preferably tea tree essential oil, clove essential oil, and/or spearmint essential oil.

In a further formulation, the constituents include thyme essential oil in an amount that is less than 1.0 weight % based on the total weight of the oral-care rinse composition, lemongrass essential oil in an amount that is less than 1.0 weight % based on the total weight of the oral-care rinse composition, and peppermint essential oil in an amount that is less than 1.0 weight % based on the total weight of the oral-care rinse composition. The water in the formulation is the primary constituent and is present in an amount that is greater than 95 weight % based on the total weight of the oral-care rinse composition, greater than 97 weight % based on the total weight of the oral-care rinse composition, or greater than 98 weight %.

These formulations work well for reducing the growth of plaque on teeth, preventing stains to teeth, and whitening teeth. Oral-Rinse Formulation #1 and #2 works very well to freshen breath for regular users. Formulation #3 (discussed below) also helps to further address halitosis. The addition of tea tree oil had a significant impact on the duration of time the formula was effective for breath freshening. Daily use is preferred with a serving of approximately 1 Tablespoon (about 15 ml) swished vigorously in the mouth and gargled for 30-60 seconds. The formulations can be used several times day, as needed.

### Oral-Rinse Formulation #3

Oral-rinse Formulation #3 has also been used to reduce dental plaque, oral malodor, and of stains to the teeth. Oral-rinse Formulation #3 includes water, thyme essential oil, lemongrass essential oil and peppermint essential oil as the primary constituents. As noted above, the essential oils have densities that are slightly lower than the density of water and they average about 0.9 g/ml, which has been used for the calculations below for Formulation #3.

TABLE 3
Oral-rinse Formulation #3.

| | | | | | Relative Weight | |
|---|---|---|---|---|---|---|
| Constituent | Amount | | Weight | | Percentage | |
| Water | 295.74 ml | | 295.74 | g | 99.44 | weight % |
| | (10 fl. oz.) | | | | | |
| Thyme Essential Oil | 0.5 | ml | 0.45 | g | 0.15 | weight % |
| Lemongrass Essential Oil | 0.5 | ml | 0.45 | g | 0.15 | weight % |
| Peppermint Essential Oil | 0.6 | ml | 0.54 | g | 0.18 | weight % |
| Tea Tree Essential Oil | 0.25 | ml | 0.23 | g | 0.08 | weight % |
| Total 297.41 g | 297.59 | ml | 297.41 | g | | |

In another embodiment based on Formulation #3, the formulation includes an amount of thyme essential oil in an amount of from 0.10 to 0.20 weight % based on the total weight of the oral-care rinse composition, lemongrass essential oil in an amount of from 0.10 to 0.20 weight % based on the total weight of the oral-care rinse composition, peppermint essential oil in an amount of from 0.15 to 0.25 weight % based on the total weight of the oral-care rinse composition, and tea tree essential oil in an amount of from 0.05 to 0.10 weight % based on the total weight of the oral-care rinse composition. The formulation includes water as the primary constituent in an amount that is greater than 95 weight % based on the total weight of the oral-care rinse composition, greater than 97 weight % based on the total weight of the oral-care rinse composition, or greater than 99 weight % based on the total weight of the oral-care rinse composition. The water may be the only constituent other than the essential oils.

In another embodiment, the oral rinse formulation includes about 99.4 weight % of water, about 0.15 weight % of thyme essential oil, about 0.15 weight % of lemongrass essential oil, about 0.18 weight % of peppermint essential oil, and of about 0.08 weight % of tea tree essential oil.

In another embodiment that is based on Formulation #3, the oral rinse formulation so greater than 95.0 weight % of water, about 0.15-65 weight % of thyme essential oil, about 0.15-65 weight % of lemongrass essential oil, about 0.15-0.65 weight % of peppermint essential oil, and of about 0.05-0.20 weight % of tea tree essential oil.

In the embodiments based on the Formulation #3, there is preferably more peppermint essential oil than the other essential oils. For example, a weight ratio of the peppermint essential oil to the thyme essential oil is about 6:5. A weight ratio of the peppermint essential oil to the lemongrass essential oil may also be about 6:5. Further, there is preferably less tea tree oil than the other essential oils. For example, the weight ratio of the thyme essential oil to the tea tree essential oil is about 2:1. A weight ratio of the lemongrass essential oil to the tea tree essential oil is about 2:1.

Instead of or in addition to the tea tree oil, formulations based on Formulation #3 may include other essential oils, such as clove essential oil or spearmint essential oil. These collective amount of the tea tree essential oil, clove essential oil, and/or spearmint essential oil is less than 0.2 weight % based on the total weight of the oral-care rinse composition. Tea tree adds powerful breath freshening for halitosis. Clove essential oil is intended to be in a formulation for tooth sensitivity or pre-exam/pre-cleaning to reduce pain and swelling (similar to Sensodyne® toothpaste). Spearmint essential oil is preferably not a replacement for peppermint oil, but acts as a flavorant, and can be added to Formulations #1, #2 and #3 for a more stimulating and freshening mouth-feel.

As noted above, formulations based on Formulation #3 may include other constituents, such as an emulsifier and/or a solubilizing agent (e.g., glycerin or lecithin), to help maintain the essential oils in suspension in the water. The emulsifier and/or the solubilizing agent is preferably present in amount that is less than 0.15 weight % based on the total weight of the oral-care rinse composition, less than 0.10 weight % based on the total weight of the oral-care rinse composition, or less than 0.05 weight % based on the total weight of the oral-care rinse composition. As noted above, the formulations do not require an emulsifier or a solubilizing agent, as the formulations can be shaken in a bottle or container before use to distribute the oils evenly within the water.

In yet a further implementation, the oral-care rinse formulation comprises water, and combinations of thyme essential oil, lemongrass essential oil, peppermint essential oil, tea tree essential oil, clove essential oil, and/or spearmint essential oil. The water is present in an amount that is greater than 97 or 99 weight % based on the total weight of the oral-care rinse composition. The essential oils are present in an amount that is preferably less than 3 weight % based on the total weight of the oral-care rinse composition. In this embodiment, the thyme essential oil, lemongrass essential oil, and peppermint essential oil are preferably present in higher amounts than the tea tree essential oil, clove essential oil, and/or spearmint essential oil.

Oral-Rinse Formulation #3 is preferably used twice a day (or as needed) with about 1 Tablespoon (about 15 ml) per serving, which is then swished in the mouth and gargled for 30-60 seconds.

In a preferred embodiment of the formulations of the present invention, there is no additional alcohol, dyes, abrasive agents, fluoride, or other additives.

### Methods of Using Oral-Rinse Formulations

Formulations #1, #2, and #3 and the additional formulations based on Formulations #1, #2, and #3 can be used as a method of neutralizing malodor in the mouth. Formulations #1, #2, and #3 and additional formulations based on Formulations #1, #2, and #3 can also be used as a method of reducing the growth of plaque on teeth. Formulations #1, #2, and #3 2 and additional formulations based on Formulations #1, #2, and #3 can also be used as a method of preventing stains to teeth. Formulations #1, #2, and #3 and additional formulations based on Formulations #1, #2, and #3 can also be used as a method of whitening teeth. In short, these formulations can be used for enhancing oral hygiene.

The formulations in accordance with the present invention may have wider pharmacological health benefits for regular users as the oils in these formulas are currently known to have anti-viral and antifungal properties, which, in addition to traditional benefits of oral hygiene for health and cosmetic purposes, may reduce fungal infections such as thrush and viral infections such as Herpes Simplex 1.

While various examples of the present disclosure have been described above, it should be understood that they have been presented by way of example only, and not limitation. Numerous changes to the disclosed examples can be made in accordance with the disclosure herein without departing from the spirit or scope of the disclosure. Thus, the breadth and scope of the present disclosure should not be limited by any of the above described examples. Rather, the scope of the disclosure should be defined in accordance with the following claims and their equivalents.

Although the disclosure has been illustrated and described with respect to one or more implementations, equivalent alterations and modifications will occur to others skilled in the art upon the reading and understanding of this specification. In addition, while a particular feature of the disclosure may have been disclosed with respect to only one of several implementations, such feature(s) may be combined with one or more other features of the other implementations as may be desired and advantageous for any given or particular application.

The terminology used herein is for the purpose of describing particular examples only and is not intended to be limiting of the disclosure. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art to which this disclosure belongs. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

1-45. (canceled)

46. An oral-care rinse composition, comprising:
water in amount that is greater than 95 weight % based on the total weight of the oral-care rinse composition;
thyme essential oil in an amount of from 0.15 to 0.65 weight % based on the total weight of the oral-care rinse composition;
lemongrass essential oil in an amount of from 0.15 to 0.65 weight % based on the total weight of the oral-care rinse composition; and
peppermint essential oil in an amount of from 0.15 to 0.65 weight % based on the total weight of the oral-care rinse composition,
wherein the oral-care rinse composition is alcohol-free and configured to be applied into an oral cavity of a user.

47. The oral-care rinse composition of claim 46, wherein the thyme essential oil is present in the amount of about 0.4 to 0.5 weight % based on the total weight of the oral-care rinse composition, the peppermint essential oil is present in the amount of about 0.4 to 0.5 weight % based on the total weight of the oral-care rinse composition, and the lemongrass essential oil is present in the amount of about 0.4 to 0.5 weight % based on the total weight of the oral-care rinse composition.

48. The oral-care rinse composition of claim 46, wherein the water is in amount that is greater than 98 weight % based on the total weight of the oral-care rinse composition.

49. The oral-care rinse composition of claim 48, further including an emulsifier and/or a stabilizing agent.

50. The oral-care rinse composition of claim 46, further including one or more additional essential oils in a collective amount that is less than 0.5 weight % based on the total weight of the oral-care rinse composition, the one or more additional essential oils additional essential oil being tea tree essential oil, clove essential oil, and/or spearmint essential oil.

51. The oral-care rinse composition of claim 46, wherein the water is the only constituent other than the essential oils.

52. An oral-care rinse composition, comprising:
water in amount that is greater than 98 weight % based on the total weight of the oral-care rinse composition;
thyme essential oil in an amount of about 0.4 to 0.5 weight % based on the total weight of the oral-care rinse composition;
lemongrass essential oil in an amount of about 0.4 to 0.5 weight % based on the total weight of the oral-care rinse composition; and
peppermint essential oil in an amount of about 0.4 to 0.5 weight % based on the total weight of the oral-care rinse composition.

53. The oral-care rinse composition of claim 52, wherein the water is the only constituent other than the essential oils.

54. The oral-care rinse composition of claim 52, further including an emulsifier or a stabilizing agent.

55. The oral-care rinse composition of claim 52, wherein the oral-care rinse composition is free from added alcohol.

56. The oral-care rinse composition of claim 52, further including one or more additional essential oils in a collective amount that is less than 0.5 weight % based on the total weight of the oral-care rinse composition.

57. The oral-care rinse composition of claim 56, wherein the one or more additional essential oils additional essential oil being tea tree essential oil, clove essential oil, and/or spearmint essential oil.

58. An oral-care rinse composition, comprising:
water in amount that is greater than 97 weight % based on the total weight of the oral-care rinse composition; and
a combination of essential oils that includes one or more of thyme essential oil, lemongrass essential oil, peppermint essential oil, tea tree essential oil, clove essential oil, and/or spearmint essential oil, wherein the combination of essential oils being in a collective amount that is between 1.0 weight % and 3.0 weight % based on the total weight of the oral-care rinse composition.

59. The oral-care rinse composition of claim 58, wherein the combination of essential oils includes at least thyme essential oil, lemongrass essential oil, and peppermint essential oil.

60. The oral-care rinse composition of claim 59, wherein the thyme essential oil is present in an amount that is less than 1.0 weight % based on the total weight of the oral-care rinse composition, the lemongrass essential oil is present in an amount that is less than 1.0 weight % based on the total weight of the oral-care rinse composition, and the peppermint essential oil is present in an amount that is less than 1.0 weight % based on the total weight of the oral-care rinse composition.

61. The oral-care rinse composition of claim 60, wherein the thyme essential oil is present in an amount of about 0.15 to 0.65 weight % based on the total weight of the oral-care rinse composition, the lemongrass essential oil is present in an amount of about 0.15 to 0.65 weight % based on the total weight of the oral-care rinse composition, and the peppermint essential oil is present in an amount of about 0.15 to 0.65 weight % based on the total weight of the oral-care rinse composition.

62. The oral-care rinse composition of claim 60, wherein the oral-care rinse composition is free from added alcohol and added chemicals.

63. The oral-care rinse composition of claim 60, wherein the water is the only constituent other than the essential oils.

64. The oral-care rinse composition of claim 60, further including one or more additional essential oils in a collective amount that is less than 0.5 weight % based on the total weight of the oral-care rinse composition, the one or more additional essential oils additional essential oil being tea tree essential oil, clove essential oil, and/or spearmint essential oil.

65. The oral-care rinse composition of claim 58, wherein the oral-care rinse composition is free from added alcohol.
